(19) United States
(12) Patent Application Publication
(10) Pub. No.: US 2011/0112347 A1
(43) Pub. Date: May 12, 2011

(54) PROCESS TO PREPARE AN OLEFIN-CONTAINING PRODUCT OR A GASOLINE PRODUCT

(76) Inventors: Robert Van Den Berg, Amsterdam (NL); Jeroen Van Westrenen, Amsterdam (NL); Andrew Leslie Ciiewter, Amsterdam (NL); Ferry Winter, Amsterdam (NL)

(21) Appl. No.: 12/988,918

(22) PCT Filed: Apr. 23, 2009
(86) PCT No.: PCT/EP2009/054920

§ 371 Date: Dec. 13, 2010

(30) Foreign Application Priority Data
Apr. 24, 2008 (EP) 08155079.0
Dec. 22, 2008 (EP) 08172609.3

(51) Int. Cl. C07C 1/24

(52) U.S. Cl. 585/324

(57) ABSTRACT

Process to prepare an olefin-containing product or a gasoline product from a solid carbonaceous feedstock by performing the steps of (a) feeding an oxygen comprising gas and the carbonaceous feedstock to a burner firing into a reactor vessel, which burner is preferably positioned horizontal, (b) performing a partial oxidation of the carbonaceous feedstock in said burner to obtain a stream of hot synthesis gas and a liquid slag, (c) cooling the hot synthesis gas by direct contacting with a liquid water-containing cooling medium, (e) performing a water shift reaction on at least part of the synthesis gas, to obtain a synthesis gas effluent, (g) performing an oxygenate synthesis using the synthesis gas effluent of step (e), to obtain a methanol and/or dimethylether containing oxygenate effluent and a first liquid water-rich by-product, (h) converting the oxygenate effluent to an olefin-containing product or a gasoline product and a second liquid water-rich by-product, wherein at least part of the first and/or second liquid water-rich by-product is used in step c), forming at least part of the liquid water-containing cooling medium.

The present invention relates to improvements relating to a solid carbonaceous feed to olefins process.

Alkylalcohols and alkylethers, such as methanol and/or dimethylether, are useful feedstock for preparing olefins in so-called oxygenate-to-olefins processes, or as feedstock for preparing gasoline in a so-called oxygenate-to-gasoline process. An oxygenate-to-olefins process can convert e.g. methanol and/or dimethylether over a catalyst to a product stream that is typically rich in lower olefins, including ethene, propene, as well as butenes, pentenes, hexenes, and also higher olefins and other hydrocarbons and some by-products. The oxygenate feedstock can be obtained from synthesis gas, also referred to as syngas. Synthesis gas is a gas comprising carbon monoxide (CO), hydrogen (H₂) and optionally carbon dioxide (CO₂). Optionally, syngas may also include methane (CH₄), ethane, propane, heavier hydrocarbons, or other compounds. A particularly interesting source of synthesis gas is from the gasification of a solid carbonaceous feedstock such as coal.

A process for converting methanol and/or dimethylether to lower olefins is known from, e.g., WO-A 2006/020083, incorporated herein by reference. This document describes a process wherein syngas and/or methanol is converted in a first step in the presence of a first catalyst to an effluent stream comprising dimethylether and water. A weight majority of the dimethylether is separated from a weight majority of the water in the effluent stream. The separated dimethylether is subsequently converted in a second step to light olefins and water in the presence of a second catalyst.

As water is formed in both the first and second reaction steps of the known process, it is said to be advantageous to provide an integrated system for removing water from the reaction system while recycling as much of the residual dimethylether and residual methanol as possible within the process for forming a light olefin product from syngas and/or methanol. In one embodiment a water removal unit is provided, which receives a first effluent stream from the first reaction step and one or more water containing streams separated from the effluent of the second reaction step. The water removal unit serves for separating residual oxygenate components such as residual dimethylether and residual methanol from the water received therein. Waste water streams obtained in the known process are preferably directed to a water treatment facility.

The separation of water containing minimum concentrations of methanol and dimethylether from the effluents of the reaction contributes significantly to the cost and overall complexity of the process.

A variety of processes is known for converting methanol and/or dimethylether to an olefin-containing product. Such processes are generally referred as to as oxygenate to olefins (OTO) processes. In an OTO reaction process, an oxygenate feedstock is converted in the presence of a molecular sieve catalyst composition into an product containing one or more olefins, preferably including light olefins, in particular ethylene and/or propylene. One such process is described in WO-A 2006/020083, incorporated herein by reference, in particular in paragraphs [0116]-[0135]. Another, preferred, process is described in WO 2007/135052. Processes integrating the production of oxygenates from synthesis gas and their conversion to light olefins are described in US2007/0203380A1 and US2007/0155999A1.

The reaction product from an OTO process typically comprises water, which is separated from an olefin-rich product component. Often the reaction product still comprises unreacted oxygenate feedstock, which can form part of the separated water stream.

WO-A-2006/070018 describes a process wherein preferably coal is converted into a synthesis gas by means of partial oxidation. Part of the synthesis gas is subjected to a catalytic water shift process step and combined with non-shifted synthesis gas. The resulting mixture is used to perform a Fischer-Tropsch synthesis to obtain a paraffinic product. In this publication reference is made to the well-known gasification processes for coal.

Examples of well-known coal gasification processes are described in U.S. Pat. No. 4,836,146 and in WO-A-2004/005438. U.S. Pat. No. 4,836,146 describes a gasification system for a solid particulate comprising a gasification reactor and a synthesis gas cooling vessel. In this publication a method and apparatus is described for controlling rapping of the heat exchange surfaces as present in the separate cooling vessel. Rapping is required to avoid deposits to accumulate on the surfaces of the heat exchangers.

A problem with the syngas cooler of WO-A-2004/005438 and U.S. Pat. No. 4,836,146 is that the heat exchanging surfaces introduce a large complexity to the design of said apparatuses. Furthermore extensive measures like rapping are required to avoid deposits to accumulate on the heat exchanger surfaces. Another problem is that the heat exchanging surfaces are even more vulnerable to fouling from feedstocks with for instance a high alkaline content. There is thus a desire to process high alkaline feedstocks as well as a desire to provide more simple processes. This especially wherein the synthesis gas is used in an OTO process, which in itself is already a complicated process.

EP-A-0400740 describes a process wherein syngas is produced by gasification of solid fuel in a reactor vessel equipped with tangential burners. The obtained slag flows downwardly while the syngas flows upwardly and is quenched in a single quench section located above the reactor.

The afore discussed gasification reactors have in common that the synthesis gas as produced flows substantially upwards and the slag flows substantially downwards relative to the gasification burners as present in said reactors. Thus, all these reactors have an outlet for slag, which is separate from the outlet for synthesis gas. These reactors have the advantage that large capacities per reactor are achievable.

WO 2006/117355 discloses a method of producing synthesis gas comprising CO, CO₂, and H₂ from a carbonaceous stream by partial oxidation in a gasification reactor, wherein water is injected in a quenching section in the form of a mist.

It is an object of the present invention to provide an improved process for preparing an olefin-containing product from a solid carbonaceous feedstock using the ‘high capacity gasification reactors’ as described above.

In accordance with the invention there is provided a process to prepare an olefin-containing product or a gasoline product from a solid carbonaceous feedstock by performing the steps of

(a) feeding an oxygen comprising gas and the carbonaceous feedstock to a burner firing into a reactor vessel, which burner is preferably positioned horizontal,
(b) performing a partial oxidation of the carbonaceous feedstock in said burner to obtain a stream of hot synthesis gas and a liquid slag,
(c) cooling the hot synthesis gas by direct contacting with a liquid water-containing cooling medium,
(e) performing a water shift reaction on at least part of the synthesis gas, to obtain a synthesis gas effluent,
(g) performing an oxygenate synthesis using the synthesis gas effluent of step (e), to obtain a methanol and/or dimethylether containing oxygenate effluent and a first liquid water-rich by-product,
(h) converting the oxygenate effluent to an olefin-containing product or a gasoline product and a second liquid water-rich by-product,
wherein at least part of the first and/or second liquid water-rich by-product is used in step c), forming at least part of the liquid water-containing cooling medium.

An advantage of the claimed process is that the synthesis gas obtained after the cooling step (c) and step (d) comprises a sufficient amount of water to perform a water shift reaction of step (e). A further advantage is that it is not needed to perform a sophisticated separation of methanol and/or dimethylether from the liquid water-rich by-product that is being recycled. By recycling these components with the water as cooling medium they can remain in the overall process as feedstock for ultimate conversion to valuable products, e.g. olefins or gasoline products. Depending on the temperature of the synthesis gas in step (c) part or all of the methanol and/or dimethylether may covert to hydrogen and carbon monoxide and form part of the synthesis gas.

Preferred solid carbonaceous feeds as used in step (a) are ash and sulphur containing feedstocks, preferably coal, biomass, for example wood, in particular torrefied wood, and waste. More preferably the solid carbonaceous feed is substantially (i.e. >90 wt. %) comprised of naturally occurring coal or synthetic (petroleum)cokes, most preferably coal. Suitable coals include lignite, bituminous coal, sub-bituminous coal, anthracite coal, and brown coal.

It will be understood that gasoline products can contain an amount of olefins. An olefin-containing product can in particular be a product containing more than 50 wt % of olefins.

In step (a) an oxygen comprising gas and the carbonaceous feedstock is fed to a burner and firing into a reactor vessel. In one embodiment the burner is positioned horizontal. In step (b) a partial oxidation of the carbonaceous feedstock in said burner is performed to obtain a stream of hot synthesis gas and a liquid slag. In one embodiment this stream of hot synthesis gas flows upwardly relative to the burner and the liquid slag flows downwardly relative to the burner. In another embodiment both synthesis gas and slag flow downwardly relative to the burner. The partial oxidation is carried out by partially combusting the carbonaceous feed with a limited volume of oxygen at a temperature normally between 800° C. and 2000° C., preferably between 1400 and 1800° C., at a pressure between 20 and 100 bar, and preferably in the absence of a catalyst. In order to achieve a more rapid and complete gasification, initial pulverisation of the solid carbonaceous feed is preferred. When the feedstock is coal the term fine particulates is intended to include at least pulverized particulates having a particle size distribution so that at least about 90% by weight of the material is less than 90 μm and moisture content is typically between 2 and 8% by weight, and preferably less than about 5% by weight.

In one embodiment, steps (a) and (b) are performed by feeding an oxygen-comprising gas and the carbonaceous feedstock to a burner firing into a combustion chamber at the upper end of the reactor vessel. Said reactor vessel is preferably provided with a quench chamber at the lower end of said vessel. The combustion chamber is fluidly connected to said quench chamber by a combustion chamber outlet opening. Step (c) is performed in said quench chamber. The walls of the combustion chamber are suitably cooled by indirect heat exchange between conduits through which a cooling medium flows and said wall. The wall itself may also be composed of said conduits. This type of wall is also referred to as a membrane wall.

The gasification is preferably carried out in the presence of oxygen comprising gas and optionally some steam, the purity of the oxygen comprising gas preferably being at least 90% by volume, nitrogen, carbon dioxide and argon being permissible as impurities. Substantially pure oxygen is preferred, such as prepared by an air separation unit (ASU). The oxygen comprising gas may contain some steam. Steam acts as moderator gas in the gasification reaction. The ratio between oxygen and steam is preferably from 0 to 0.3 parts by volume of steam per part by volume of oxygen. The oxygen used is preferably heated before being contacted with the coal, preferably to a temperature of from about 200 to 500° C.

The partial oxidation reaction is preferably performed by combustion of a dry mixture of fine particulates of the carbonaceous feed and a carrier gas with oxygen in a suitable burner. Examples of suitable burners are described in U.S. Pat. No. 48,887,962, U.S. Pat. No. 4,523,529 and U.S. Pat. No. 4,510,874.

The gasification chamber is preferably provided with one or more partial oxidation burners, wherein said burners are provided with supply means for a solid carbonaceous feed and supply means for an oxygen containing gas. The burners or burner may be directed downwardly from the roof of the combustion chamber or fire horizontally or substantially horizontally. In the case of horizontal firing or substantial horizontal firing it is preferred to have pairs of diametrically-positioned burners. With a pair of burners is here meant two burners, which are directed horizontal and diametric into the gasification chamber. This results in a pair of two burners in a substantially opposite direction at the same horizontal position. The reactor may be provided with 1 to 5 of such pairs of burners, preferably 2 to 5 of such pairs. The upper limit of the number of pairs will depend on the size of the reactor. The firing direction of the burners may be slightly tangential as for example described in EP-A-400740.

Examples of suitable carrier gasses to transport the dry and solid feed to the burners are steam, nitrogen, synthesis gas and preferably carbon dioxide. Carbon dioxide is preferred because it achieves a better selectivity to synthesis gas and avoids build-up of nitrogen in downstream gas recycle streams.

In step (c) the hot synthesis gas is cooled by direct contacting the gas with a liquid water-containing cooling medium. The direct contacting is preferably achieved by injecting the liquid water-containing cooling medium into the gaseous stream of synthesis gas or by passing the synthesis gas through a batch of the liquid water-containing cooling medium, or combinations of said methods.

In one embodiment, the hot synthesis gas which is contacted with liquid water-containing cooling medium may be cooled in two steps. A first cooling may be achieved by injecting a quench gas into the hot synthesis gas wherein the temperature is reduced from between 1400 and 1800° C. to a temperature between 500 and 900° C. Cooling with a gas quench is well known and described in for example EP-A-416242, EP-A-662506 and WO-A-2004/005438. Examples of suitable quench gases are recycle synthesis gas and steam.

A first cooling step is preferred to achieve a gas temperature below the solidification temperature of the non-gaseous components present in the hot synthesis gas.

More preferably the first cooling is performed by injecting a mist of liquid droplets into the gas flow as will be described in more detail below. The use of the liquid mist as compared to a gas quench is advantageous because of the larger cooling capacity of the mist. Moreover, at the high temperatures of the hot syngas from step b), oxygenates, in particular methanol, decomposes into synthesis gas components. The liquid may be any liquid having a suitable viscosity in order to be atomized. Non-limiting examples of the liquid to be injected are a hydrocarbon liquid, a waste stream etc. Preferably the liquid comprises at least 50% water. Most preferably the liquid is substantially comprised of water (i.e. >80 vol %, in particular >90 vol %). Examples of suitable sources of water are the wastewater, also referred to as black water, as obtained in the synthesis gas scrubber or the process condensate of the downstream water shift reactor is used as the liquid.

In another embodiment, step (c) is performed by injecting the liquid water-containing cooling medium as a spray into the synthesis gas as discharged into the quench chamber as described above. Alternatively the synthesis gas may flow through a diptube fluidly connected to the outlet opening of the combustion chamber and partly submerged in a water bath as present at the lower end of the quench chamber. In such a reactor it is preferred to cool the inner walls of the diptube by a stream of a liquid comprising the liquid water-containing cooling medium, which flows downwardly and along the inner wall of the diptube. Combinations of such diptube cooling and spraying are possible.

According to the invention, part or all of the liquid water-containing cooling medium is formed by water-rich by-product as obtained in step (g) and/or (h), that is being recycled to step c), to form at least part of the liquid water-containing cooling medium. This water condensate is the water portion as obtained as by-product when performing step (g) and/or (h) and will typically contain water as the predominant component and water soluble compounds as produced in the conversion of syngas to olefins. These compounds are for example alcohols, carboxylic acids and other oxygenates. These compounds will at least in part decompose at the elevated temperature conditions when contacted with the hot synthesis gas. By using this water in this manner a costly and complicated waste water process for the water by-product of step (g) and/or (h) is avoided. In particular a significant concentration of methanol in the recycled liquid water-rich by-product can be accepted, such as 20 wt % or less, in particular 10 wt % or less, more in particular 5 wt % or less, even more in particular 2 wt % or less. The same advantageous upper concentration limits can apply to total oxygenates. When the methanol concentration is high, such as above 0.5 wt %, or above 1 wt %, it is preferred to use the water-rich by-product for quenching at high temperatures such as during the first cooling step, high enough so that the methanol decomposes into syngas components and remains in the overall process to produce olefins.

In one embodiment, the recycled liquid water-rich by-product contains at least 0.5 wt % of methanol, in particular at least 1 wt %, more in particular at least 2 wt %, even more in particular at least 5 wt %, still more in particular at least 8 wt %. Also, a concentration of dimethylether in the recycled liquid water-rich by-product can be accepted. In one embodiment, the recycled liquid water-rich by-product contains at least 0.1 wt % of dimethylether, in particular at least 0.5 wt %.

Overall, the recycled liquid water-rich by-product can contain at least 0.5 wt % oxygenates, in particular at least 1 wt %, more in particular at least 3 wt %, even more in particular at least 5 wt %, even more in particular at least 8 wt %, based on the total recycled liquid water-rich by-product. These oxygenates are not wasted by recycling in this manner.

Preferably in the process, the liquid water-containing cooling medium has a temperature of at most 50° C. below the bubble point of water at the pressure of the hot synthesis gas, in particular when the liquid water-containing cooling medium is injected water-containing mist.

With the term ‘mist’ is meant that the liquid is injected in the form of small droplets. If water is the predominant component of the liquid water-containing cooling medium, constituting more than 50 wt % thereof, preferably more than 80 wt % thereof, then preferably more than 80%, more preferably more than 90%, of the water is in the liquid state. Preferably the injected mist has a temperature of at most 50° C. below the bubble point at the prevailing pressure conditions at the point of injection, particularly at most 15° C., even more preferably at most 10° C. below the bubble point. To this end, if the injected liquid is predominantly water, it usually has a temperature of above 90° C., preferably above 150° C., more preferably from 200° C. to 230° C. The temperature will obviously depend on the operating pressure of the gasification reactor, i.e. the pressure of the raw synthesis as specified further below. Hereby a rapid vaporization of the injected mist is obtained, while cold spots are avoided. As a result the risk of ammonium chloride deposits and local attraction of ashes in the gasification reactor is reduced.

Further it is preferred that the mist comprises droplets having a diameter of from 50 to 200 μm, preferably from 100 to 150 μm. Preferably, at least 80 vol. % of the injected liquid is in the form of droplets having the indicated sizes.

To enhance quenching of the hot synthesis gas, the mist is preferably injected with a velocity of 30-90 m/s, preferably 40-60 m/s.

Also it is preferred that the mist is injected with an injection pressure of at least 10 bar above the pressure of the raw synthesis gas as present in the gasification reactor, preferably from 20 to 60 bar, more preferably about 40 bar, above the pressure of the raw synthesis gas. If the mist is injected with an injection pressure of below 10 bar above the pressure of the raw synthesis gas, the droplets of the mist may become too large. The latter may be at least partially offset by using an atomisation gas, which may e.g. be N₂, CO₂, steam or synthesis gas, more preferably steam or synthesis gas. Using atomisation gas has the additional advantage that the difference between injection pressure and the pressure of the raw synthesis gas may be reduced to a pressure difference of between 5 and 20 bar.

Further it has been found especially suitable when the mist is injected in a direction away from the gasification reactor, or said otherwise when the mist is injected in the flow direction of the synthesis gas, more preferably under an angle. Hereby no or less dead spaces occur which might result in local deposits on the wall of the apparatus in which step (c) is performed. Preferably the mist is injected under an angle of between 30-60°, more preferably about 45°, with respect to a plane perpendicular to the longitudinal axis of the conduit or vessel in which the cooling takes place.

According to a further preferred embodiment, the injected mist is at least partially surrounded by a shielding fluid. Herewith the risk of forming local deposits is reduced. The shielding fluid may be any suitable fluid, but is preferably selected from the group consisting of an inert gas such as N₂ and CO₂, synthesis gas, steam and a combination thereof.

The partly cooled synthesis gas having a temperature of between 500 and 900° C. is further cooled in a second cooling, to a temperature of below 500° C., according to the process of step (c) by direct contacting the gas with liquid water-containing cooling medium. Recycled liquid water-rich by-product from steps g) and/or h) is preferably used for this purpose only when they do not contain high concentrations of methanol, such as less than 0.5 wt %, since at the lower temperatures in the second cooling step the methanol will not decompose. The direct cooling with liquid water may suitably be performed by injecting a mist of liquid water into the synthesis gas as described above. According to an especially preferred embodiment, the total amount of water injected is selected such that the cooled synthesis gas comprises at least 40 vol. % H₂O, preferably from 40 to 60 vol. % H₂O, more preferably from 45 to 55 vol. % H₂O. The cooled synthesis gas, preferably having this water content, is preferably subjected to a dry-solids removal step to at least partially remove dry ash. Preferred solids removal units are cyclones or filter units as for example described in EP-A-551951 and EP-A-1499418.

In the above-described second cooling step the partly cooled synthesis gas may also be cooled such that the amount of water added relative to the raw synthesis gas is even higher than the preferred ranges above. This embodiment is referred to as a so-called overquench. In an overquench type process the amount of water added is such that not all liquid water will evaporate and some liquid water will remain in the cooled raw synthesis gas. Such a process is advantageous because a dry solid removal step can be omitted. In such a process the raw synthesis gas leaving the cooling vessel is saturated with water. The weight ratio of the raw synthesis gas and water injection is suitably between 1:1 to 1:4.

Overquench type process conditions may be achieved by injecting a large amount of water into the flow path of the synthesis gas, by passing the flow of synthesis gas through a water bath positioned at the lower end of the cooling vessel or combinations of these measures.

It has been found that herewith the capital costs can be substantially lowered, as no further or significantly less addition of steam in an optional downstream water shift conversion step is necessary. With capital costs is here meant the capital costs for steam boilers to generate said steam which would otherwise be added.

The synthesis gas as obtained in step (c) may be submitted to an optional water scrubbing process step (d), wherein solids are separated from the cooled synthesis gas. Such a process step is well known and therefore not described in detail. The water scrubbing step generates a water stream containing solids, also referred to as black water.

In step (e) the gaseous stream as obtained in step (c) or (d) is shift converted by at least partially converting CO into CO₂, thereby obtaining a CO depleted stream. In this step the H₂/CO ratio of the synthesis gas is increased from a lower level, typically below 1 and especially from between 0.3-0.6 for coal-derived synthesis gas to a higher value preferably above 1. The higher H₂/CO ratio is preferred to perform step (g) in the most optimal manner. Stoichiometrically, two moles of H2 and one mole of CO form one mole of methanol. The optimal H₂/CO ratio for step (g) can be dependent on the type of catalyst used in step (g).

The water shift conversion reaction as performed in step (e) is well known in the art. Generally, water, usually in the form of steam, is mixed with the gaseous stream to form carbon dioxide and hydrogen. The catalyst used can be any of the known catalysts for such a reaction, including iron, chromium, copper and zinc. Copper on zinc oxide is a known shift catalyst.

The catalytic water shift conversion reaction of step (e) provides a hydrogen enriched, often highly enriched, synthesis gas, possibly having a H₂/CO ratio above 3, more suitably above 5, preferably above 7, more preferably above 15, possibly 20 or even above.

In order to arrive at the desired H₂/CO ratio for performing step (g) it is preferred to perform step (e) only on part of the gaseous stream obtained in step (d). In this preferred embodiment the scrubbed synthesis gas of step (d) is divided into at least two sub-streams, one of which undergoes step (e) to obtain a first CO depleted stream. This first CO depleted stream is combined with the second sub-stream to form a second CO depleted stream having the desired H₂/CO ratio for performing step (g). Alternatively the cooled synthesis gas of step (c) may be split into at least two streams. Each stream is subjected to an optional scrubbing step (d) separately. At least one stream is subjected to a step (e) to obtain a first CO depleted stream and at least one stream is not subjected to a step (e) to obtain the second sub-stream.

If desired one or more of the sub-stream(s) which are not subjected to step (e) could be used for other parts of the process rather than being combined with the converted sub-stream(s). Preferably part of such sub-stream is used for steam or power generation.

Hydrogen is preferably prepared from part of a CO depleted stream, more preferably from the first CO depleted stream. Hydrogen is preferably prepared in a Pressure Swing Adsorption (PSA) unit, a membrane separation unit or combinations of these. The hydrogen manufactured in this way can then be used as the hydrogen source in a possible further hydroprocessing step wherein the hydrocarbon products as made in step (g) are used as feed. This arrangement reduces or even eliminates the need for a separate source of hydrogen, e.g. from an external supply, which is otherwise commonly used where available.

The division of the gaseous stream as obtained in step (d), or optionally in step (c), into sub-streams can be such so as to create any desired H₂/CO ratio following their recombination. Any degree or amount of division is possible. Where the gaseous stream are divided into two sub-streams, the division into the sub-streams could be in the range 80:20 to 20:80 by volume, preferably 70:30 to 30:70 by volume, depending upon the desired final H₂/CO ratio. Simple analysis of the H₂/CO ratios in the second CO depleted stream and knowledge of the desired ratio for subsequent process steps, in particular step (g), allows easy calculation of the division.

The simple ability to change the degree of division into the sub-streams also provides a simple but effective means of accommodating variation in the H₂/CO ratio in the gaseous stream as obtained in step (b) which variations are primarily due to variation in feedstock quality. With feedstock quality is here meant especially the hydrogen and carbon content of the original carbonaceous feedstock, for example, the ‘grade’ of coal. Certain grades of coal generally having a higher carbon content will, after gasification of the coal, provide a greater production of carbon monoxide, and thus a lower H₂/CO ratio. However, using other grades of coal means removing more contaminants or unwanted parts of the coal, such as ash and sulphur and sulphur-based compounds. The ability to change the degree of division of the synthesis gas stream into the sub-streams allows the process to use a variety of feedstocks, especially ‘raw’ coal, without any significant re-engineering of the process or equipment to accommodate expected or unexpected variation in such coals.

Preferably, after step (e) an additional step (f) of separating sulphur compounds, carbon dioxide and other possible impurities from the shifted gas to obtain a purified synthesis gas effluent, is performed, and step (g) uses the purified synthesis gas effluent for the oxygenate synthesis.

Suitably the synthesis gas is subjected to a CO₂ recovery system thereby obtaining a CO₂ rich stream and a CO₂ poor stream and wherein the CO₂ poor stream is used in step (g). The CO₂ rich stream may be used as the CO₂ containing transport gas in step (a).

In one embodiment it is preferred to remove part of the CO₂ as is present in the synthesis gas intended for use in step (g). Part of the CO₂ is preferably used in step (a) in an embodiment wherein CO₂ is used as carrier gas. Excess CO₂ is preferably stored in subsurface reservoirs or used more preferably for enhanced oil or gas recovery or enhanced coal bed methane recovery. Excess CO₂ may also be sequested by mineral carbonation such as for example described in WO-A-02/085788.

The CO₂ recovery system is preferably a combined carbon dioxide/hydrogen sulfide removal system, preferably wherein the removal system uses a physical solvent process. The CO₂ recovery may be performed on the CO-depleted stream or alternatively on the second CO-depleted stream. More preferably the CO₂ recovery from the sub-stream, which stream is not being subjected to step (e), is performed separately from the CO₂ recovery from the first CO depleted stream before said streams are combined.

On an industrial scale there are chiefly two categories of absorbent solvents, depending on the mechanism to absorb the acidic components: chemical solvents and physical solvents. Each solvent has its own advantages and disadvantages as to features as loading capacity, kinetics, regenerability, selectivity, stability, corrosivity, heat/cooling requirements etc.

Chemical solvents which have proved to be industrially useful are primary, secondary and/or tertiary amines derived alkanolamines. The most frequently used amines are derived from ethanolamine, especially monoethanol amine (MEA), diethanolamine (DEA), triethanolamine (TEA), diisopropanolamine (DTPA) and methyldiethanolamine (MDEA).

Physical solvents which have proved to be industrially suitable are cyclo-tetramethylenesulfone and its derivatives, aliphatic acid amides, N-methylpyrrolidone, N-alkylated pyrrolidones and the corresponding piperidones, methanol, ethanol and mixtures of dialkylethers of polyethylene glycols.

A well-known commercial process uses an aqueous mixture of a chemical solvent, especially DIPA and/or MDEA, and a physical solvent, especially cyclotetramethylene-sulfone. Such systems show good absorption capacity and good selectivity against moderate investment costs and operational costs. They perform very well at high pressures, especially between 20 and 90 bara.

The physical absorption process is preferred and is well known to the man skilled in the art. Reference can be made to e.g. Perry, Chemical Engineerings' Handbook, Chapter 14, Gas Absorption. The liquid absorbent in the physical absorption process is suitably methanol, ethanol, acetone, dimethylether, methyl i-propyl ether, polyethylene glycol or xylene, preferably methanol. This process is based on carbon dioxide and hydrogen sulfide being highly soluble under pressure in the methanol, and then being readily releasable from solution when the pressure is reduced as further discussed below. This high pressure system is preferred due to its efficiency, although other removal systems such as using amines are known. The physical absorption process is suitably carried out at low temperatures, preferably between −60° C. and 0° C., preferably between −30 and −10° C.

The physical absorption process is carried out by contacting the light products stream in a counter-current upward flow with the liquid absorbent. The absorption process is preferably carried out in a continuous mode, in which the liquid absorbent is regenerated. This regeneration process is well known to the man skilled in the art. The loaded liquid absorbent is suitably regenerated by pressure release (e.g. a flashing operation) and/or temperature increase (e.g. a distillation process). The regeneration is suitably carried out in two or more steps, preferably 3-10 steps, especially a combination of one or more flashing steps and a distillation step.

The regeneration of solvent from the process is also known in the art. Preferably, the present invention involves one integrated solvent regeneration tower. Further process conditions are for example described in DE-A-2610982 and DE-A-4336790.

Preferably the synthesis gas is subjected to one or more further removal systems prior to using said stream in step (g). These removal systems may be guard or scrubbing units, either as back-up or support to the CO₂/H₂S removal system, or to assist in the reduction and/or removal of other contaminants such as HCN, NH₃, COS and H₂S, metals, carbonyls, hydrides or other trace contaminants.

In step (g) an oxygenate synthesis using the synthesis gas effluent of step (e), or optionally (f), is performed, to obtain a methanol and/or dimethylether containing oxygenate effluent and a first liquid water-rich by-product.

Step (g) can be or include a syngas to methanol conversion process. This can be any methanol synthesis process, in particular any conventional methanol synthesis process, including e.g. batch processes and, preferably, continuous processes.

In general, methanol synthesis can be described by the following reactions:

CO+2H₂→CH₃OH

CO₂+3H₂→CH₃OH+H₂O

By the second reaction, in particular when the syngas contains CO₂, water is produced as by-product in the methanol-containing effluent, which is suitably separated. According to the present invention, this separation does not need to be conducted to achieve high water purity, since residual methanol in the separated water can advantageously be recycled to step (c) of the process. The water is also referred to as third liquid water-rich by-product in the present description and claims.

A methanol synthesis process is for example described in WO 2006/020083, incorporated by reference, in particular in paragraphs [0069] to [0086]. As stated there, the syngas input to the methanol synthesis reactor suitably has a molar ratio of hydrogen (H₂) to carbon oxides (CO+CO₂) in the range of from about 0.5:1 to about 20:1, preferably in the range of from about 2:1 to about 10:1. In another embodiment, the syngas has a molar ratio of hydrogen (H₂) to carbon monoxide (CO) of at least 2:1. In a further embodiment of the present invention the molar ratio is in the range from about 1:1 to about 10:1. Carbon dioxide is optionally present in an amount of not greater than 50% by weight, based on total weight of the syngas.

Also as stated there, the stoichiometric molar ratio is sufficiently high so as maintain a high yield of methanol, but not so high as to reduce the volume productivity of methanol. Preferably, the syngas fed to the methanol synthesis has a stoichiometric molar ratio (i.e., a molar ratio of H₂:(2CO+3CO₂)) of from about 1.0:1 to about 2.7:1, more preferably from about 1.1 to about 2.0, more preferably a stoichiometric molar ratio of from about 1.2:1 to about 1.8:1.

The syngas can contain CO₂ and CO at a molar ratio of from about 0.5 to about 1.2, preferably from about 0.6 to about 1.0.

WO 2006/020083 moreover states suitable catalysts, refers to suitable processes for making methanol synthesis catalysts, states suitable temperature and pressure ranges for a methanol synthesis process, as well as suitable gas hourly space velocities for a continuous process, all incorporated herein by reference.

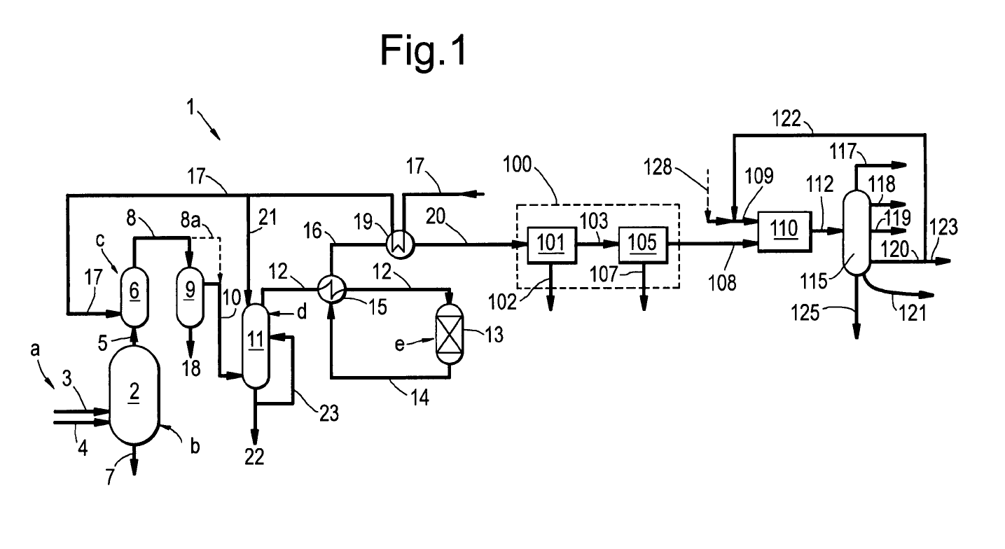

WO 2006/020083 also discusses refining of crude methanol to make a methanol product, in paragraphs [0087]-[0094], incorporated herein by reference. As important element of the refining is the removal of by-product water from this process, as already discussed above. With reference to FIG. 1 of WO 2006/020083, the synthesis of methanol from syngas stream 106, which can be obtained in accordance with the present invention from a solid carbonaceous feedstock, and the refining in a separation zone is discussed on page 29 line 8-page 30 line 13, incorporated herein by reference. The water stream 118 in FIG. 1 can be used as water-rich by-product that is recycled to step c), to form at least part of the liquid water-containing cooling medium.

The methanol thus produced can form the oxygenate effluent from step (g) that is converted in step (h).

In one embodiment, the oxygenate effluent from step (g) and converted in step (h) comprises dimethylether, preferably at least 0.1 mol % of the oxygenates therein, more preferably at least 0.3 mol %. To this end step (g) of the process preferably contains a step of converting methanol and/or syngas to dimethylether.

Processes to convert syngas and/or methanol to dimethylether are discussed in WO 2006/020083, in particular in section III, incorporated herein by reference. Dimethylether can be produced directly from syngas; or in a two-step process from methanol first produced from syngas, or from methanol and syngas together. Along all routes, a water-containing by-product is produced which can be recycled to step (c) according to the present invention, wherein it is acceptable that methanol and/or dimethylether are contained in the recycle stream.

In a preferred embodiment, in a first step, (g1), of step (g), at least part of the synthesis gas effluent is converted to a methanol containing effluent and a third liquid water-rich by-product (such as described hereinabove), and then, in step (g2), at least part of the methanol containing effluent is converted to a dimethylether containing effluent and a fourth liquid water-rich by-product. The third and fourth liquid water-rich by-products form part of the first liquid water-rich by-product as defined above, wherein preferably at least part of the first water-rich by-product is recycled to step c).

In an especially preferred embodiment at least part of the fourth liquid water-rich by-product is recycled to step c), and this can be the only water-containing by-product stream from steps (g) and (h) that is being recycled.

The conversion of methanol to dimethylether is known in the art. This conversion is an equilibrium reaction. In the conversion the alcohol is contacted at elevated temperature with a catalyst. In EP-A 340 576 a list of potential catalysts are described. These catalysts include the chlorides of iron, copper, tin, manganese and aluminium, and the sulphates of copper, chromium and aluminium. Also oxides of titanium, aluminium or barium can be used. Preferred catalysts include aluminium oxides and aluminium silicates. Alumina is particularly preferred as catalyst, especially gamma-alumina. Although the methanol may be in the liquid phase the process is preferably carried out such that the methanol is in the vapour phase. In this context the reaction is suitably carried out at a temperature of 140 to 500° C., preferably 200 to 400° C., and a pressure of 1 to 50 bar, preferably from 8-12 bar. In view of the exothermic nature of the conversion of methanol to dimethylether the conversion of step a) is suitably carried out whilst the reaction mixture comprising the first catalyst is being cooled to maximise dimethylether yield.

The ratio of dimethylether and methanol in the product stream (dimethylether containing effluent) of step (g2) may vary between wide ranges. Suitable ranges include a dimethylether to methanol weight ratio of 0.5:1 to 100:1, preferably from 2:1 to 20:1. Suitably the reaction is led to equilibrium. This includes that the dimethylether to methanol weight ratio may vary from 2:1 to 6:1. Evidently, the skilled person may decide to influence the equilibrium by applying different reaction conditions and/or by adding or withdrawing any of the reactants.

It may be advantageous to maintain a pH of at least 7 in the hot dimethylether containing effluent. Therefore, this liquid water-containing stream is suitably enriched with a base. Enriching with a base provides an elegant solution to get rid of by-products, in particular hydrocarbonaceous by-products, that are formed in the production of dimethylether from methanol, by removal with the liquid water-containing stream. In particular the enrichment of the dialkylether product stream with a base forms products, in particular neutralization products, which can be removed with the water-containing stream. In one embodiment a pH of at least 7 is maintained in the hot dialkylether product stream, in particular in a liquid water-containing fraction of the dialkylether product stream. In order to enrich the dialkylether product stream with a base, the base is suitably contacted with or added to the dialkylether product stream (or a fraction thereof), such that a pH of from 7 to 12 is achieved in a liquid water-containing fraction of the dialkylether product stream. Such a base can be sodium or potassium hydroxide, or any other alkali metal or alkaline earth metal bases or mixtures thereof. The base may be added to the hot dimethylether containing product stream or in any preceding stream.

Other suitable processes to convert syngas to methanol and/or dimethylether are described in e.g. US2007/0203380A1 and US2007/0155999A1.

In step (h) the oxygenate effluent from step (g) is converted to an olefin-containing product and a second liquid water-rich by-product. The olefins manufacture from oxygenates, in particular methanol and/or dimethylether is known in the art. Suitable processes to convert a dimethylether-containing feedstock to light olefins are have already been referred to above, for example are described in WO 2006/020083, section IV, incorporated herein by reference.

The catalysts described therein are also suitable for the process of the present invention. Such catalysts preferably include molecular sieve catalyst compositions. Excellent molecular sieves are silicoaluminophosphates (SAPO), such as SAPO-17, -18, -34, -35, -44, but also SAPO-5, -8, -11, -20, -31, -36, -37, -40, -41, -42, -47 and -56. Alternatively, the olefin manufacture may be accomplished by the use of an aluminosilicate catalyst. Suitable catalysts include those containing a zeolite of the ZSM group, in particular of the MFI type, such as ZSM-5, the MTT type, such as ZSM-23, the TON type, such as ZSM-22. Other suitable zeolites are for example e zeolites of the STF-type, such as SSZ-35, the SFF type, such as SSZ-44 and the EU-2 type, such as ZSM-48. Preferably, the dimethylether-rich feed is converted over a catalyst comprising a one-dimensional zeolite that has 10-membered ring channels. Such a zeolite is more preferably selected from the group consisting of aluminosilicates of the MTT and TON type and mixtures thereof. The present invention can be of particular advantage for processes wherein the olefin conversion is accomplished over a catalyst comprising a one-dimensional zeolite having 10-membered ring channels, in particular a zeolite of the MTT and/or TON type, since it has been found that the hydrothermal deactivation of the catalyst is reduced when the dimethylether-rich feed stream contains no or only minor amounts, i.e. <5% wt, of water.

Advantageously, the catalyst comprises one or more zeolites, of which at least 50% wt has one-dimensional 10-membered ring channels, which are not intersected by other channels, such as zeolites of the MTT and/or TON type. In a particularly preferred embodiment the catalyst comprises in addition to one or more one-dimensional zeolites having 10-membered ring channels, such as of the MTT and/or TON type, a more-dimensional zeolite, in particular of the MFI type, more in particular ZSM-5, since this further zeolite (molecular sieve) has a beneficial effect on the stability of the catalyst in the course of the process and under hydrothermal conditions. The second molecular sieve having more-dimensional channels has intersecting channels in at least two directions. So, for example, the channel structure is formed of substantially parallel channels in a first direction, and substantially parallel channels in a second direction, wherein channels in the first and second directions intersect. Intersections with a further channel type are also possible. Preferably the channels in at least one of the directions are 10-membered ring channels. Especially when the olefins manufacture is carried out over a catalyst containing MTT or TON type aluminosilicates, it may be advantageous to add an olefin-containing co-feed together with the dimethylether-rich feed to the reaction zone when the latter feed is introduced into this zone. It has been found that the catalytic conversion of dimethylether to olefins is enhanced when an olefin is present in the contact between dimethylether and catalyst. Therefore, suitably, an olefinic co-feed is added to the reaction zone together with the dimethylether-rich feed when one reaction zone is employed.

A specially preferred process for use in step (h) of the present invention is described in WO 2007/135052 A1, incorporated herein by reference, which process can advantageously convert methanol and/or dimethylether containing oxygenate feed to olefins over a catalyst comprising one-dimensional zeolite having 10-membered ring channels.

In a particular embodiment, step (h) of the process comprises the steps of

h1) reacting an oxygenate feedstock comprising oxygenate species having an oxygen-bonded methyl group, preferably methanol and/or dimethylether, and an olefinic co-feed, in the presence of a oxygenate conversion catalyst comprising at least 50 wt %, based on total molecular sieve in the oxygenate conversion catalyst, of a molecular sieve having one-dimensional 10-membered ring channels, to prepare an olefinic reaction effluent, wherein the olefinic co-feed preferably comprises less than 10 wt % of C5+ hydrocarbon species;
h2) fractionating the olefinic reaction effluent to obtain at least a light olefinic fraction comprising ethylene, a heavier olefinic fraction comprising C4 olefins and preferably less than 10 wt % of C5+ hydrocarbon species, and the second liquid water-rich by-product;
h3) recycling at least part of the heavier olefinic fraction obtained in step b) as recycle stream to step a), to form at least part of the olefinic co-feed; and
h4) withdrawing at least part of the light olefinic fraction as olefinic product.

Like for hot dimethylether containing effluent discussed hereinbefore it may be advantageous to maintain a pH of at least 7 in the second liquid water-rich fraction that can be used in step (c) of the process, and this can be achieved by enriching with a base, in the ways discussed hereinabove for hot dimethylether containing effluent.

In special embodiments, at least 70 wt % of the olefinic co-feed in step h1), during normal operation, is formed by the recycle stream of step h3), preferably at least 90 wt %, more preferably at least 99 wt %, and most preferably the olefinic co-feed is during normal operation formed by the recycle stream. The heavier olefinic fraction can comprise at least 50 wt % of C4 olefins, and at least a total of 70 wt % of C4 hydrocarbon species. The heavier olefinic fraction can also comprise propylene. The olefinic reaction effluent can comprises 10 wt % or less, preferably 5 wt % or less, more preferably 1 wt % or less, of C6-C8 aromatics, based on total hydrocarbons in the effluent. At least one of the olefinic co-feed, and the olefinic recycle stream, can in particular comprise less than 5 wt % of C5+ olefins, preferably less than 2 wt % of C5+ olefins, based on total hydrocarbons in the olefinic co-feed.

Optimum light olefins yield are obtained in preferred embodiments, wherein step h1) is conducted at a temperature of more than 450° C., preferably at a temperature of 460° C. or higher, more preferably at a temperature of 480° C. or higher.

In a special embodiment, the oxygenate conversion catalyst comprises more than 50 wt %, preferably at least 65 wt %, based on total molecular sieve in the oxygenate conversion catalyst, of the one-dimensional molecular sieve having 10-membered ring channels.

The oxygenate conversion catalyst can comprise at least 1 wt %, based on total molecular sieve in the oxygenate conversion catalyst, of a further molecular sieve having more-dimensional channels, preferably at least 5 wt %, more preferably at least 8 wt %, and furthermore can comprise less than 35 wt % of the further molecular sieve, preferably less than 20 wt %, more preferably less than 18 wt %, still more preferably less than 15 wt %. Preferably the further molecular sieve is a MFI-type molecular sieve, in particular zeolite ZSM-5. A preferred MFI-type zeolite has a Silica-to-Alumina ratio SAR of at least 60, preferably at least 80, more preferably at least 100, even more preferably at least 150.

Preferably, molecular sieves in the hydrogen form are used in the oxygenate conversion catalyst, e.g., HZSM-22, HZSM-23, and HZSM-48, HZSM-5. Preferably at least 50% w/w, more preferably at least 90% w/w, still more preferably at least 95% w/w and most preferably 100% of the total amount of molecular sieve used is in the hydrogen form. When the molecular sieves are prepared in the presence of organic cations the molecular sieve may be activated by heating in an inert or oxidative atmosphere to remove organic cations, for example, by heating at a temperature over 500° C. for 1 hour or more. The zeolite is typically obtained in the sodium or potassium form. The hydrogen form can then be obtained by an ion exchange procedure with ammonium salts followed by another heat treatment, for example in an inert or oxidative atmosphere at a temperature over 500° C. for 1 hour or more. The molecular sieves obtained after ion-exchange are also referred to as being in the ammonium form.

The molecular sieve can be used as such or in a formulation, such as in a mixture or combination with a so-called binder material and/or a filler material, and optionally also with an active matrix component. Other components can also be present in the formulation. If one or more molecular sieves are used as such, in particular when no binder, filler, or active matrix material is used, the molecular sieve itself is/are referred to as oxygenate conversion catalyst. In a formulation, the molecular sieve in combination with the other components of the mixture such as binder and/or filler material is/are referred to as oxygenate conversion catalyst.

It is desirable to provide a catalyst having good mechanical or crush strength, because in an industrial environment the catalyst is often subjected to rough handling, which tends to break down the catalyst into powder-like material. The latter causes problems in the processing. Preferably the molecular sieve is therefore incorporated in a binder material. Examples of suitable materials in a formulation include active and inactive materials and synthetic or naturally occurring zeolites as well as inorganic materials such as clays, silica, alumina, silica-alumina, titania, zirconia and aluminosilicate. For present purposes, inactive materials of a low acidity, such as silica, are preferred because they may prevent unwanted side reactions which may take place in case a more acidic material, such as alumina or silica-alumina is used.

The preferred embodiment of step (h) described hereinabove is preferably performed in a reactor system comprising a fluidized bed or moving bed, e.g. a fast fluidized bed or a riser reactor system. In one embodiment a reactor system comprising two or more serially arranged riser reactor stages is used, to obtain a riser reactor effluent from each stage, wherein each riser reactor stage comprises a single riser reactor or a plurality of parallel riser reactors, such that at least part of the riser reactor effluent of a preceding riser reactor stage is fed into a subsequent riser reactor stage.

In one embodiment, step (h1) is performed in a reactor system comprising a plurality of sequential reaction zones, and wherein oxygenate is added to at least two of the sequential reaction zones.

When multiple reaction zones are employed, an olefinic co-feed is advantageously added to the part of the dimethylether-rich feed that is passed to the first reaction zone.

The olefinic co-feed may contain one olefin or a mixture of olefins. Apart from olefins, the olefinic co-feed may contain other hydrocarbon compounds, such as for example paraffinic, alkylaromatic, aromatic compounds or a mixture thereof. Preferably the olefinic co-feed comprises an olefinic fraction of more than 50 wt %, more preferably more than 60 wt %, still more preferably more than 70 wt %, which olefinic fraction consists of olefin(s). The olefinic co-feed can consist essentially of olefin(s).

Any non-olefinic compounds in the olefinic co-feed are preferably paraffinic compounds. If the olefinic co-feed contains any non-olefinic hydrocarbon, these are preferably paraffinic compounds. Such paraffinic compounds are preferably present in an amount in the range from 0 to 50 wt %, more preferably in the range from 0 to 40 wt %, still more preferably in the range from 0 to 30 wt %.

By an olefin is understood an organic compound containing at least two carbon atoms connected by a double bond. A wide range of olefins can be used. The olefin can be a mono-olefin, having one double bond, or a poly-olefin, having two or more double bonds. Preferably olefins present in the olefinic co-feed are mono-olefins.

The olefin(s) can be a linear, branched or cyclic olefin. Preferably olefins present in the olefinic co-feed are linear or branched olefins.

Preferred olefins have in the range from 2 to 12, preferably in the range from 3 to 10, and more preferably in the range from 4 to 8 carbon atoms.

Examples of suitable olefins that may be contained in the olefinic co-feed include ethene, propene, butene (one or more of 1-butene, 2-butene, and/or iso-butene (2-methyl-1-propene)), pentene (one or more of 1-pentene, 2-pentene, 2-methyl-1-butene, 2-methyl-2-butene, 3-methyl-1-butene, and/or cyclopentene), hexene (one or more of 1-hexene, 2-hexene, 3-hexene, 2-methyl-1-pentene, 2-methyl-2-pentene, 3-methyl-1-pentene, 3-methyl-2-pentene, 4-methyl-1-pentene, 4-methyl-2-pentene, 2,3-dimethyl-1-butene, 2,3-dimethyl-2-butene, 3,3-dimethyl-1-butene, methylcyclopentene and/or cyclohexene), heptenes, octenes, nonenes and decenes. The preference for specific olefins in the olefinic co-feed may depend on the purpose of the process.

In a preferred embodiment the olefinic co-feed preferably contains olefins having 4 or more carbon atoms (i.e. C₄+ olefins), such as butenes, pentenes, hexenes and heptenes. More preferably the olefinic fraction of the olefinic co-feed comprises at least 50 wt % of butenes and/or pentenes, even more preferably at least 50% wt of butenes, and most preferably at least 90 wt % of butenes. The butene may be 1-, 2-, or iso-butene. Most conveniently it is a mixture thereof. More preferably, the olefinic co-feed that is added to the reaction zone is a by-product of the olefin conversion step e) of the present process which by-product contains 4 to 7, preferably just 4, carbon atoms and which is recycled to the reaction zone. These relatively higher olefins tend to facilitate the conversion of methanol and/or dimethylether to olefins such as propylene and ethylene.

The preferred molar ratio of oxygenate in the oxygenate feedstock to olefin in the olefinic co-feed depends on the specific oxygenate used and the number of reactive oxygen-bonded alkyl groups therein. Preferably the molar ratio of oxygenate to olefin in the total feed lies in the range of 10:1 to 1:10, more preferably in the range of 5:1 to 1:5 and still more preferably in the range of 3:1 to 1:3.

The reaction conditions of the olefin manufacture include those that are mentioned in WO-A 2006/020083. Hence, a reaction temperature of 200 to 1000° C., preferably from 250 to 750° C., and a pressure from 0.1 kPa (1 mbar) to 5 MPa (50 bar), preferably from 100 kPa (1 bar) to 1.5 MPa (15 bar), are suitable reaction conditions. For the preferred process described in WO 2007/135052, reference is made to suitable and preferred process conditions as disclosed therein.

The reaction of the oxygenate in step (h) may be carried out in a single reaction zone, as described in WO-A 2006/020083. However, it is preferred that the conversion takes place in several reaction zones into each of which oxygenate feed is fed. Accordingly, part of the oxygenate feed is passed to multiple reaction zones comprising a first reaction zone and one or more subsequent reaction zones, where the oxygenate is converted to an olefin. Evidently, the multiple reaction zones may be operated in parallel. However, it is preferred that the multiple reaction zones are arranged in series. In that way at least part or substantially all of the product of the previous reaction zone is forwarded to the subsequent reaction zone. Also the catalyst of the previous reaction zone may be forwarded to the subsequent reaction zone together with its product, i.e. the entire effluent from a previous reaction zone can be forwarded. Hence, the number of reaction zones may suitably vary from 1 to 6, preferably from 2 to 4.

Processes to convert an oxygenate effluent to a gasoline type product are well known. A known example is the ExxonMobil Methanol to Gasoline (MTG) Process. For example WO-A-0129152 describes a process for selectively converting the oxygenate effluent to normally liquid boiling range C5+ hydrocarbons in a single step. The process comprises, contacting the feed under oxygenate conversion conditions with a catalyst comprising a unidimensional 10-ring zeolite, e.g., one selected from the group consisting of ZSM-22, ZSM-23, ZSM-35, ZSM-48, ZSM-57, and ferrierite, at temperatures below 350° C. and oxygenate pressures above 40 psia (276 kPa); and recovering a normally liquid boiling range C5+ hydrocarbons-rich product stream, for example gasoline. When a gasoline product is produced in accordance with the present invention, the liquid rich by-product obtained in such a process is referred to as the second liquid water-rich by-product and can be recycled to step (c).

Further details of the invention will now be described with reference to the drawings.

FIG. 1 schematically shows schematically a first process scheme for performing a method according the present invention.

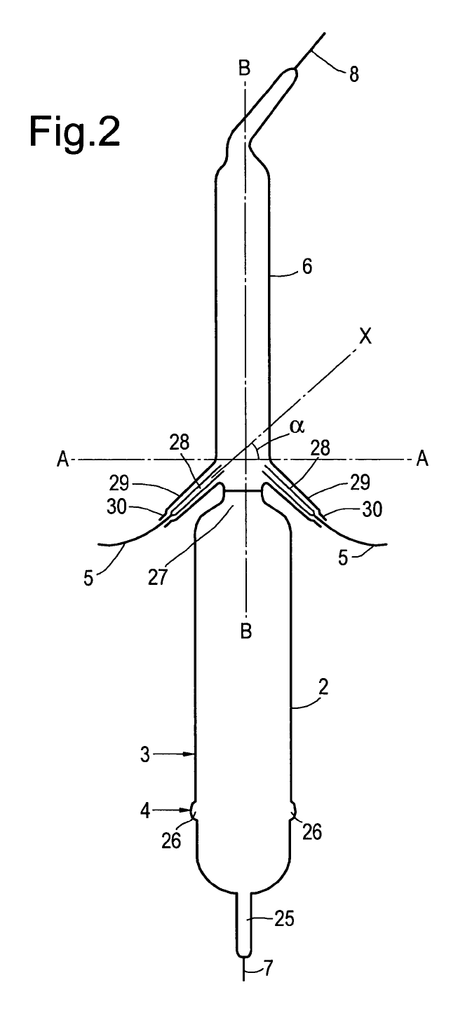

FIG. 2 schematically shows schematically a longitudinal cross-section of a first gasification reactor used in the system according to the present invention, in particular as depicted in FIG. 1.

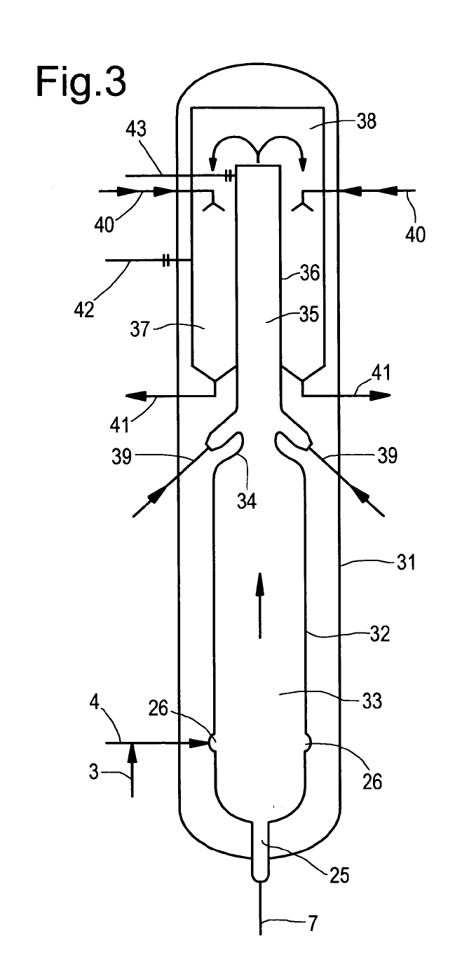

FIG. 3 schematically shows schematically a longitudinal cross-section of a first reactor, which may be used in the system according to the present invention.

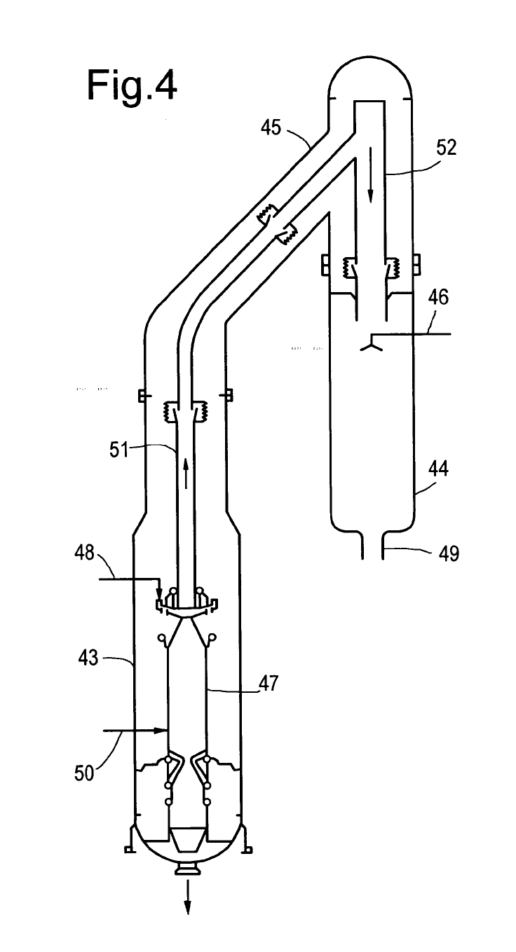

FIG. 4 schematically shows schematically a gasification reactor system for performing the two-step cooling method making use of a downstream separate vessel.

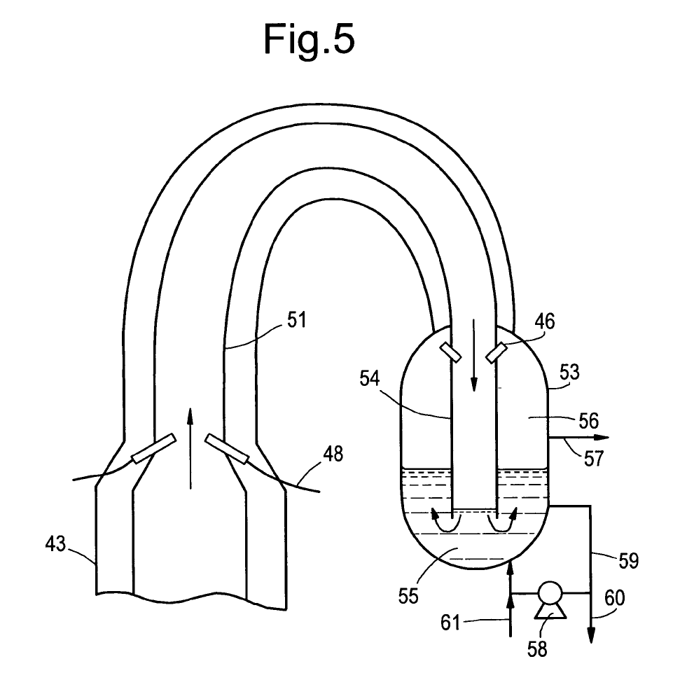

FIG. 5 schematically shows schematically a preferred embodiment for the gasification reactor system of FIG. 4.

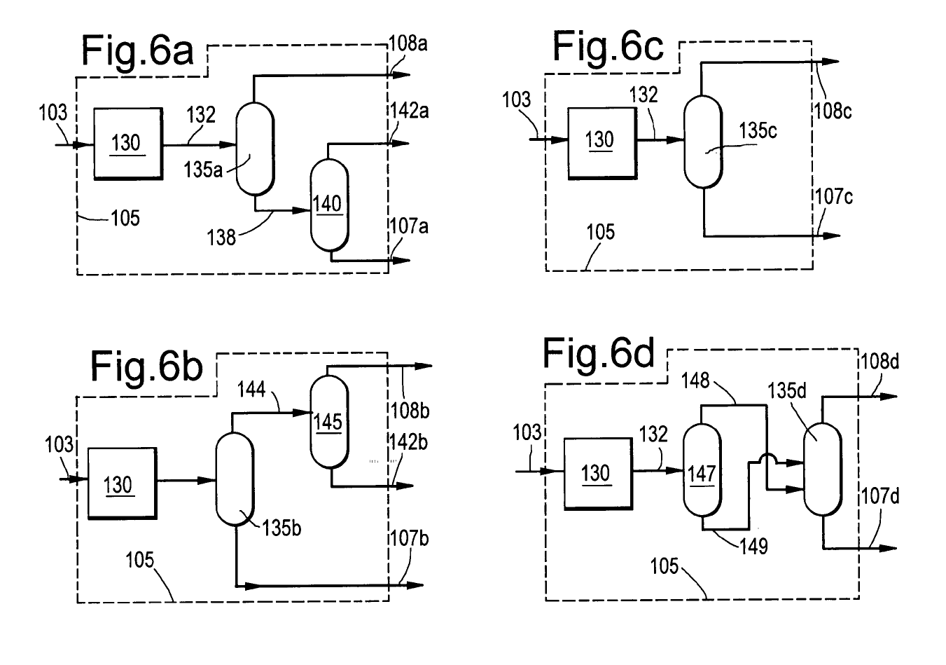

FIG. 6a,b,c,d shows schematically various embodiments of the prereactor unit of FIG. 1.

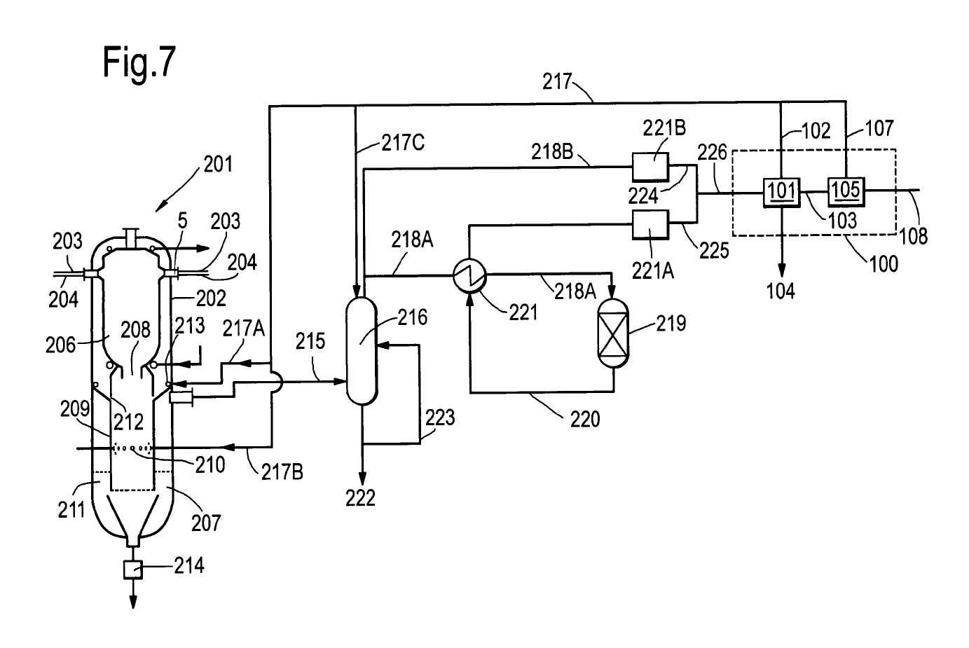

FIG. 7 schematically shows part of a second process scheme for performing a method according the present invention

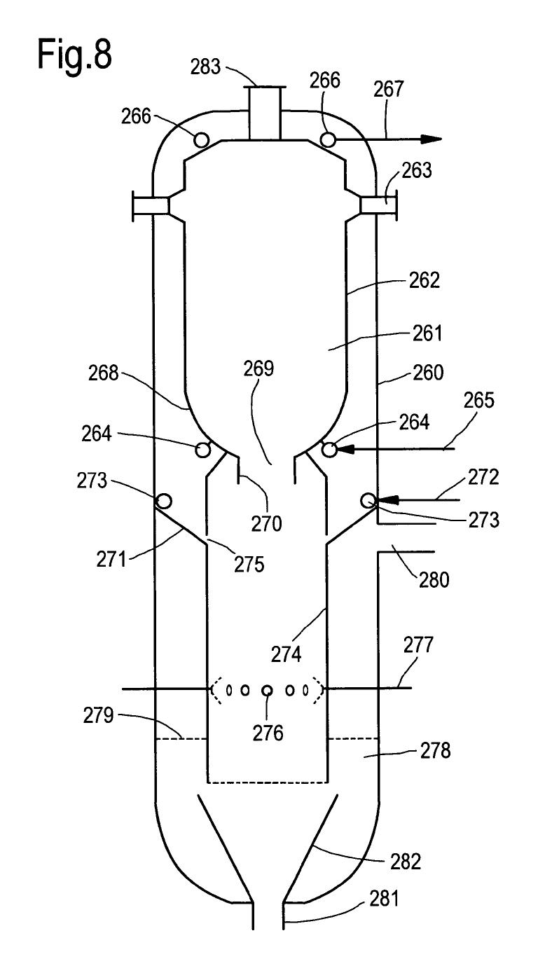

FIG. 8 schematically shows schematically a longitudinal cross-section of a gasification reactor of FIG. 7.

Same reference numbers as used below refer to the same or similar structural elements.

Reference is made to FIG. 1. FIG. 1 schematically shows an embodiment of a system 1 for producing an olefin-containing product via synthesis gas. In a gasification reactor 2 a carbonaceous stream and an oxygen containing stream may be fed via lines 3, 4, respectively.

The solid carbonaceous feed is at least partially oxidised in the gasification reactor 2, thereby obtaining a synthesis gas and a slag. To this end usually several burners (not shown) are present in the gasification reactor 2. The produced hot synthesis gas is fed via line 5 to a cooling section 6; herein the hot synthesis gas is cooled by contacting with liquid water-containing cooling medium 17. Into the cooling section 6 liquid water-containing cooling medium is injected via line 17 as will be further discussed in FIG. 2 below. The slag drops down and is drained through line 7 for optional further processing.

As shown in the embodiment of FIG. 1, the cooled synthesis gas leaving the cooling section 6 is further processed. To this end, it is fed via line 8 into a dry solids removal unit 9 to at least partially remove dry ash in the cooled synthesis gas. Dry ash is removed form the dry solids removal unit via line 18. In an overquench mode of operation said dry-solids removal unit 9 is omitted and the cooled synthesis gas is directly fed to a wet gas scrubber 11 via line 8a and 10.

After the dry solids removal unit 9 the synthesis gas is fed via line 10 to a wet gas scrubber 11. Part of the scrubbed gas is subsequently fed via line 12 to a shift converter 13 to react at least a part of the water with CO to produce CO₂ and H₂, thereby obtaining a first CO-depleted stream in line 14. Waste water from gas scrubber 11 is removed via line 22 and optionally partly recycled to the gas scrubber 11 via line 23.

It has surprisingly been found that according to the present invention, the vol. % water of the stream leaving the cooling section 6 in line 8 is already such that the capacity of the wet gas scrubber 11 may be substantially lowered, resulting in a significant reduction of capital expenses.

Further improvements are achieved when the raw synthesis gas in line 12 is heated in a heat exchanger 15 against the shift converted synthesis gas in line 14 that is leaving the shift converter 13.

Further it is preferred that energy contained in the stream of line 16 leaving heat exchanger 15 is used to warming up the water in line 17 to be injected in cooling section 6. To this end, the stream in line 16 may be fed to an indirect heat exchanger 19, for indirect heat exchange with the stream in line 17.

As shown in the embodiment in FIG. 1, the stream in line 14 is first fed to the heat exchanger 15 before entering the indirect heat exchanger 19 via line 16. However, the person skilled in the art will readily understand that the heat exchanger 15 may be dispensed with, if desired, or that the stream in line 14 is first fed to the indirect heat exchanger 19 before heat exchanging in heat exchanger 15. If desired the heated stream in line 17 may also be partly used as a feed (line 21) to the gas scrubber 11.

The CO depleted stream in line 20 can be fed to a carbon dioxide and/or hydrogen sulphide removal system (not shown), and is then fed to oxygenate synthesis unit 100. In the embodiment of the oxygenate synthesis unit 100 shown, the synthesis gas effluent from line 20 is first converted in the methanol synthesis unit 101 to a methanol-containing effluent in line 103 and a third water-containing by-product, which is separated in the unit 101 and removed as stream 102. There can also be obtained other by-products, such as fusel oils described in WO A 2006/020083, which can be separated and removed separately (not shown). In the embodiment shown here, the methanol-containing effluent in line 103 is at least partly converted to dimethylether in the prereactor unit 105, which also forms part of the oxygenate synthesis unit 100. Water is formed in this reaction in prereactor unit 105, which is separated in the unit 105, and a fourth liquid water-rich by-product stream 107 is removed. The stream 108 contains dimethylether and typically some methanol, as well as some water. The main function of the prereactor 105 is to lower the water load on the downstream oxygenate-to-olefins reactor 110. Since further water will be formed in that reactor, in one embodiment a high purity in oxygenates in stream 108 is not needed, so a simple separation of the bulk of water from in the prereactor effluent is sufficient. This is also true for the fourth water-containing by-product stream 107, which is preferably recycled into line 17 in accordance with the present invention. Oxygenates still contained in this water stream are retained within the process and not wasted. The third and fourth water-containing by-product streams 102 and 107 together form the first water-containing by-product stream. Either one or both streams 102 and 107 can be recycled partly or fully to line 17.

The prereactor unit can also be omitted, and the oxygenate stream 108 can in this case be substantially formed of the methanol-containing effluent 103.

The oxygenate stream, e.g. methanol and/or dimethylether containing effluent, is fed to the reactor system 110 via line 108. In the embodiment shown, an olefinic co-feed, as specified herein above, is fed to the reactor system as well, via line 109. In the reactor system 110, the dimethylether containing effluent and the olefinic co-feed are allowed to react in the presence of a oxygenate conversion catalyst as specified herein above, to prepare an olefinic reaction effluent in line 112.

The olefin-containing product in line 112 is sent to a fractionation section schematically shown as 115. In this embodiment the fractionation section is shown to produce an ethylene-rich product stream in line 118 as light olefinic fraction, and a C4-olefin-rich stream in line 120, as heavier olefinic fraction, and further a lighter stream in overhead line 117 comprising lighter contaminants such as methane and/or carbon oxides, a propylene-rich stream in line 119 and a C5+ hydrocarbon rich stream in line 121.

At least part of the heavier olefinic fraction in line 120 is recycled via line 122 to an inlet of the reactor system 110, to form at least part of the olefinic co-feed. If desired, part of the heavier olefinic fraction can be withdrawn via line 123. If this is merely a small bleed stream, such as less than 10 wt % or in particular less than 5 wt %, substantially all of the heavier olefinic fraction is considered to be recycled. To the recycle stream other components can be blended, such as from the propylene rich stream 119 or from the C5+ hydrocarbon-rich stream 121. The latter can increase yield of lower olefins, but is less desired because it does so at the cost of ethylene selectivity. The second water-containing by-product stream 125 is also obtained from the separation system 115, and can be recycled to fully or in part to line 17. Recycling this stream can be interesting when the OTO reaction has not converted the oxygenates fully, and the stream 125 still contains oxygenates which can be maintained within the process to prepare olefins by recycling.

In special and not generally preferred embodiments, such as during start-up, part of the olefinic co-feed can be obtained from an external source via line 128. If that is not the case, the overall process converts oxygenate in line 108 to mainly light olefins in lines 118 and 119.

In FIG. 6, several embodiments of the prereactor unit 105 of FIG. 1 are schematically shown. Turning first to FIG. 6a, the methanol-containing effluent in line 103 is at least partly converted to dimethylether in the prereactor 130, and effluent in line 132 is fed to first separation unit or column 135a, which can be a fractionation/distillation column, that produces an first stream 108a rich in dimethylether, and optionally containing some methanol and a relatively low amount of water, and a bottom stream 138. The bottom stream is fed to a second separation unit 140, which can also be a distillation column, to produce a methanol-rich stream 142a and a water-rich bottom stream 107a.

In FIG. 6b, the first separation unit 135b is operated such that a water-rich stream 107b of sufficient quality is obtained right away, and the overhead stream 144 rich in dimethylether and containing the majority of the methanol is separated in second separation unit 145, to obtain a stream 108b rich in dimethylether, and a methanol-rich stream 142b.

The streams 142a and 142b in FIGS. 6a and 6b can e.g. be, fully or in part, recycled to the inlet of unit 105, or sent to unit 110, or even sent as recycle stream to line 17.

In a two-stage separation as in FIGS. 6a and 6b a fairly good separation between the three main components can be achieved. The water-rich stream in line 107a,b can contain less than 1 wt % of oxygenate, even less than 0.5 wt %, such as less than 0.2 wt %.

The embodiment of FIG. 6c is simpler in that only one separation unit 135c is arranged. Suitably this unit is operated such that, next to dimethylether-rich stream 108c, a water-rich stream 107c containing a sufficiently low amount of methanol and/or DME is obtained, so that it can economically be recycled to line 17. The oxygenate content will typically be higher than in the two stage embodiments discussed before, such as between 0.5 wt and 20 wt %, or between 1 wt % and 18 wt %, or between 5 wt % and 15 wt %. Recycling in this way in accordance with the invention simplifies the separation of the effluent from the DME prereactor without wasting valuable oxygenates.

The embodiment of FIG. 6d is a variant of 6c. Prior to the single separation unit 135d, the effluent 132 is sent to a flash vessel 147. In the flash vessel 147 the pressure is reduced and the product stream is cooled to below the dew point of water. The vaporous effluent from the flash vessel 147 comprises most of the dimethylether and some methanol, and leaves the flash vessel via a line 148. The liquid effluent, comprising water and methanol, leaves the flash vessel 147 via a line 149. The effluents from lines 148 and 149 are both fed into a fractionation column 135d, whereby line 149 debouches into the fractionation column 135d at a point above the location where line 148 debouches into column 135d. In fractionation column 135d the gas-liquid mixture, obtained from both streams, is separated into a liquid-rich stream 107d comprising water and less than 1% wt methanol, based on the total of water and methanol, and a vaporous dimethylether-rich stream 108d, comprising dimethylether, the majority of the methanol and typically some water.

The streams 107a,b,c,d and 108a,b,c,d in FIGS. 6a,b,c,d have the meaning as discussed with reference to FIG. 1.

An alternative suitable route to produce dimethylether from methanol is described in the paper “Dehydration of methanol to dimethylether by catalytic distillation”, An, W. et al., Canadian Fournal of Chemical Engineering (2004), vol. 82, p. 948-955.

It is within the skill of the artisan to determine the correct conditions for fractionation/separation of the mixtures as described herein. He may choose the design of the separation units such as a fractionation column, and operating conditions based on, i.a., fractionation temperature, pressure, trays, reflux and reboiler ratios, to arrive at a suitable separation.

FIG. 2 shows a longitudinal cross-section of a gasification reactor 2 used in the system 1 of FIG. 1.

The gasification reactor 2 has an inlet 3 for a solid carbonaceous stream and an inlet 4 for an oxygen containing gas.

Usually several burners (schematically denoted by 26) are present in the gasification reactor 2 for performing the partial oxidation reaction. The pair of burners is directed horizontal and diametric as shown. For reasons of simplicity, only one pair of burners 26 is shown here.

Further, the gasification reactor 2 comprises an outlet 25 for removing the slag formed during the partial oxidation reaction via line 7.

Also, the gasification reactor 2 comprises an outlet 27 for the raw synthesis gas produced, which outlet 27 is connected with the cooling section 6.

The cooling section 6 comprises a first injector 28 (connected to line 17) that is adapted for injecting a water containing stream in the form of a mist in the cooling section.

As shown in FIG. 2, the first injector in use injects the mist in a direction away from the outlet 27 of the gasification reactor 2. To this end the centre line X of the mist injected by the first injector 28 forms an angle α of between 30-60°, preferably about 45°, with respect to the plane A-A perpendicular to the longitudinal axis B-B of the cooling section 6.

Preferably, the cooling section also comprises a second injector 29 (connected via line 30 to a source of shielding gas) adapted for injecting a shielding fluid at least partially surrounding the mist injected by the at least one first injector 28. As shown in the embodiment of FIG. 2 the first injector 28 is to this end partly surrounded by second injector 29.

As already discussed above in respect of FIG. 1, the partly cooled synthesis gas leaving the cooling section 6 via line 8 may be further cooled. Examples of such further cooling are provided in FIGS. 3 and 4.

FIG. 3 illustrates a preferred gasification reactor comprising the following elements:
a pressure shell (31) for maintaining a pressure higher than atmospheric pressure;
an outlet (25) for removing the slag, preferably by means of a so-called slag bath, located in a lower part of the pressure shell (31);
a gasifier wall (32) arranged inside the pressure shell (31) defining a gasification chamber (33) wherein during operation the synthesis gas can be formed, a lower open part of the gasifier wall (32) which is in fluid communication with the outlet for removing slag (25). The open upper end (34) of the gasifier wall (32) is in fluid communication with a quench zone (35);
a quench zone (35) comprising a tubular formed part (36) positioned within the pressure shell (31), open at its lower and upper end and having a smaller diameter than the pressure shell (31) thereby defining an annular space (37) around the tubular part (36). The lower open end of the tubular formed part (36) is fluidly connected to the upper end of the gasifier wall (32). The upper open end of the tubular formed part (36) is in fluid communication with the annular space (37) via deflector space (38).

At the lower end of the tubular part (36) injecting means (39) are present for injecting a liquid or gaseous cooling medium to perform the first cooling. Preferably the direction of said injection is as described for FIG. 2 in case of liquid injections. In the annular space (37) injecting means (40) are present to inject liquid water-containing cooling medium, preferably in the form of a mist, preferably in a downwardly direction, into the partly cooled synthesis gas as it flows through said annular space (37) to perform the second cooling. FIG. 3 further shows an outlet (41) for synthesis gas is present in the wall of the pressure shell (31) fluidly connected to the lower end of said annular space (37). Preferably the quench zone is provided with cleaning means (42) and/or (43), which are preferably mechanical rappers, which by means of vibration avoids and/or removes solids accumulating on the surfaces of the tubular part and/or of the annular space respectively.

The advantages of the reactor according to FIG. 3 are its compactness in combination with its simple design. By cooling with the liquid in the form of a mist in the annular space (37) additional cooling means in said part of the reactor can be omitted which makes the reactor more simple. Preferably both via injectors (39) and injectors (40) liquid water-containing cooling medium is injected in the form of a mist as described above.

FIG. 4 illustrates an embodiment for performing the two-step cooling method making use of a separate vessel. FIG. 4 shows the gasification reactor (43) of FIG. 1 of WO-A-2004/005438 in combination with a downstream quench vessel (44) fluidly connected by transfer duct (45). The system of FIG. 4 differs from the system disclosed in FIG. 1 of WO-A-2004/005438 in that the syngas cooler (3) of said FIG. 1 of said patent publication is omitted and replaced by a simple vessel comprising means (46) to add liquid water-containing cooling medium to perform the second cooling. Shown in FIG. 4 is the gasifier wall (47), which is connected to a tubular part (51), which in turn is connected to an upper wall part (52) as present in quench vessel (44). At the lower end of the tubular part (51) injecting means (48) are present for injecting a liquid or gaseous cooling medium to perform the first cooling as illustrated in FIG. 2. Quench vessel (44) is further provided with an outlet (49) for cooled synthesis gas. FIG. 4 also shows a burner (50). The various other details of the gasification reactor (43) and the transfer duct (45) as well as the upper design of the quench vessel (44) are preferably as disclosed for the apparatus of FIG. 1 of WO-A-2004/005438.

FIG. 5 shows the upper end of gasification reactor (43) and the upper end of gasification chamber wall (47). This upper end is fluidly connected by means of connecting conduit (51) to separate cooling vessel (53). Injecting means (48) are present to inject a gaseous or liquid quenching medium in accordance with the process of the present invention.

In cooling vessel (53) a dip tube (54) is present to create a downwardly directed flow path for synthesis gas. At the upper end of the dip-tube (54) injecting means (46) are present to inject a mist of liquid water-containing cooling medium into the synthesis gas. The dip-tube is partly submerged in a water bath (55) formed by the liquid water-containing cooling medium. In use the synthesis gas will flow through water bath (55) to an annular space (56) as present between dip-tube (54) and the wall of the cooling vessel (53). From said annular space (56) the water saturated synthesis gas is discharged from said cooling vessel via conduit (57).

FIG. 5 also shows a pump (58) to recirculate water (59), providing a bleed stream (60) and a supply stream (61) for fresh water.

Reference is made to FIG. 7. FIG. 7 schematically shows a further embodiment system 201 for producing an product such as an olefin-containing product or a gasoline product via synthesis gas. In a gasification reactor 202 a carbonaceous stream and an oxygen containing stream may be fed via lines 203, 204, respectively. FIG. 1 shows a pair of burners 205 firing horizontally into a combustion chamber 206 at the upper end of the reactor 202 and a quench chamber 207 at the lower end of the reactor 2 fluidly connected by a combustion chamber outlet opening 208. The outlet opening 208 is fluidly connected to a diptube 209. Diptube 209 is provided with spray nozzles fluidly connected to a cooling water supply 217B. Diptube 209 is partly submerged in a water bath 211 as present at the lower end of the quench chamber 207. The inner walls of the diptube 209 are cooled by a stream of a liquid water cooling medium comprising as discharged like a waterfall from circular opening 212. The liquid water-containing cooling medium for this waterfall is supplied via distributor 213. Distributor 213 in turn being supplied by cooling water supply 217A.

The solid carbonaceous feed is at least partially oxidised in the gasification reactor 202, thereby obtaining a synthesis gas and a slag. The slag drops down into the water bath 211 and is drained through slag sluicing device 214 for further processing. An example of a slag sluicing device is described in EP-B-1224246, which reference is hereby incorporated by reference.

As shown in the embodiment of FIG. 7, the cooled synthesis gas leaving the quench chamber 207 is fed via line 215 to a wet gas scrubber 216. Part of the scrubbed gas is subsequently fed via line 218A to a shift converter 219 to react at least a part of the water with CO to produce CO₂ and H₂, thereby obtaining a CO-depleted stream in line 220. Waste water from gas scrubber 216 is removed via line 222 and optionally partly recycled to the gas scrubber 216 via line 223. Fresh water for scrubber 216 may be recycled water-rich by-product supplied via line 217C.

Further improvements are achieved when the raw synthesis gas in line 218A is heated in a heat exchanger 221 against the shift converted synthesis gas in line 218A that is leaving the shift converter 216.

The CO depleted stream in line 220 can be fed to a carbon dioxide and/or hydrogen sulphide removal system 221A. The remaining scrubbed synthesis gas 218B, which gas bypasses the shift reactor 219, is fed to a carbon dioxide and/or hydrogen sulphide removal system 221B. The treated gas 224 is combined with the shifted and treated synthesis gas 225, to obtain a combined synthesis gas effluent 226 having a modified hydrogen to carbon monoxide molar ratio as feedstock for oxygenate synthesis unit 100.

The embodiment of the oxygenate synthesis unit 100 shown is operated substantially as the unit 100 discussed with reference to FIG. 1. Second and third water-containing by-product streams 102 and 107 together form the first water-containing by-product stream. Either one or both streams 102 and 107 can be recycled partly or fully via line 17 to the quench chamber 7. The oxygenate stream 108, e.g. methanol and/or dimethylether containing effluent, is fed to a reactor system (not shown) for converting to an olefin-containing product or a gasoline product, wherein the second water-rich by-product is formed. The reactor system can for example be the reactor system 110 discussed with reference to FIG. 1. The second water-containing by-product stream, such as stream 125 in FIG. 1, can be recycled to fully or in part to line 217. Recycling this stream can be interesting when the OTO or oxygenate-to-gasoline reaction has not converted the oxygenates fully, and the stream 125 still contains oxygenates which can be maintained within the process to prepare olefins by recycling.

FIG. 8 shows a longitudinal cross-section of a gasification reactor 202 of FIG. 7 with somewhat more details. The reference of FIG. 8 refer to the same elements as described when discussing FIG. 7 above. FIG. 8 shows a gasification reactor 260 wherein a cooling section is part of the gasification reactor as will be described below. Reactor 260 is provided with a gasification chamber 261 as defined by a so-called membrane wall 262. Gasification chamber 261 is provided with one or more pairs of diametrically-opposed burners 263. The membrane wall is composed of vertical conduits, which are fixed together and in which, in use, a cooling medium, i.e. evaporating water, flows from a distributor 264 to a steam header 266. Distributor 264 is provided with a cooling medium supply line 265 and steam header 266 is provided with a steam discharge conduit 67. At the lower end of the tubular membrane wall a diverging frusto-conical part 268 is attached. At the lower opening 269 of said part 268 a tubular part 270, extending downwards, is provided to guide the slag and synthesis gas into a diptube 274. By having an opening 269, which is smaller than the diameter of diptube 274, one intends to avoid as much as possible that slag particles contact the inner walls of the diptube 274.

The inner walls of diptube 274 are wetted by a downwardly-moving layer of water. This layer of water is achieved by introducing water via supply ring 273. The introduced water will flow via a sloped plane 271 to circular opening 275 and further downwards along the inner wall of diptube 274.

Step (c) of the process according to the present invention is performed by introducing the liquid water-containing cooling medium via supply conduit 277, corresponding to 217B in FIG. 1, and nozzles 276 into the flow of synthesis gas. Step (c) is preferably performed by supplying the liquid water-containing cooling medium distributor 273 via supply conduit 272, corresponding to 217A in FIG. 1. In this manner the inner walls of the diptube 274 are cooled by a waterfall like stream of the liquid water cooling medium as discharged from circular opening 275. Because the temperature of the synthesis gas at the location of opening 275 is still high decomposition of the oxygenates as present in the cooling medium is most likely.

FIG. 2 further shows a water bath 278 having a surface 279. Through this water bath 278 synthesis gas will be further cooled. The cooled synthesis gas is discharged from the reactor 260 via outlet 280. Slag particles are guided via cone 282 to outlet 281. Reference number 283 may be a start-up burner or a manhole.

The person skilled in the art will readily understand that the present invention may be modified in various ways without departing from the scope as defined in the claims.

1. A process to prepare an olefin-containing product from a solid carbonaceous feedstock by performing the steps of
(a) feeding an oxygen comprising gas and the carbonaceous feedstock to a burner firing into a reactor vessel, which burner is positioned horizontal,
(b) performing a partial oxidation of the carbonaceous feedstock in said burner to obtain a stream of hot synthesis gas and a liquid slag,
(c) cooling the hot synthesis gas by direct contacting with a liquid water-containing cooling medium,
(e) performing a water shift reaction on at least part of the synthesis gas, to obtain a synthesis gas effluent,
(g) performing an oxygenate synthesis using the synthesis gas effluent of step (e), to obtain a methanol and/or dimethylether containing oxygenate effluent and a first liquid water-rich by-product, and
(h) converting the oxygenate effluent to an olefin-containing product and a second liquid water-rich by-product,
wherein at least part of the first and/or second liquid water-rich by-product is used in step c), forming at least part of the liquid water-containing cooling medium.

2. The process according to claim 1, wherein in step (b) the stream of hot synthesis gas flows upwardly relative to the burner and the liquid slag flows downwardly relative to the burner, and further including, between steps (c) and (e) the step of
(d) separating solids from the cooled synthesis gas by means of a water scrubbing process step, whereby the water shift reaction of step (e) is performed on at least part of the scrubbed synthesis gas.

3. The process according to claim 1, wherein the synthesis gas is cooled in step (c) by first cooling the gas to a temperature of between 500 and 900° C. by injecting a gaseous or liquid cooling medium into the synthesis gas and subsequently further cooling the gas in to below 500° C. by direct contacting with a liquid cooling medium.

4. The process according to claim 3, wherein the second cooling is performed by injecting the water-containing cooling medium into the synthesis gas or by passing the synthesis gas through a bath of the water-containing cooling medium, to obtain a synthesis gas having a weight ratio of synthesis gas and water-containing cooling medium of between 1:1 to 1:4 and wherein the cooled synthesis gas obtained in step (c) is directly used as feed to the scrubbing step of step (d).

5. The process according to claim 4, wherein the second cooling is performed by injecting a mist of water-containing droplets into the synthesis gas.

6. The process according to claim 3, wherein said first cooling is performed by injecting a mist of water-containing droplets into the synthesis gas.

7. The process according to claim 1, wherein in step (b) both synthesis gas and slag flow downwardly relative to the burner.

8. The process according to claim 7, wherein step (b) is performed by feeding an oxygen-comprising gas and the carbonaceous feedstock to a burner firing into a combustion chamber at the upper end of the reactor vessel and a quench chamber at the lower end of the reactor vessel fluidly connected by a combustion chamber outlet opening and wherein step (c) is performed in said quench chamber.

9. The process according to claim 8, wherein step (c) is performed by injecting the liquid water-containing cooling medium as a spray into the synthesis gas as discharged into the quench chamber.

10. The process according to claim 8, wherein the synthesis gas flows through a diptube fluidly connected to the outlet opening of the combustion chamber and partly submerged in a water bath as present at the lower end of the quench chamber, wherein the inner walls of the diptube are cooled by a stream of a liquid comprising the liquid water-containing cooling medium which flows downwardly and along the inner wall of the diptube.

11. The process according to claim 1, wherein step (g) comprises the steps of
(g1) converting at least part of the synthesis gas effluent to a methanol containing effluent and a third liquid water-rich by-product; and
(g2) converting at least part of the methanol containing effluent to a dimethylether containing effluent and a fourth liquid water-rich by-product,
wherein the third and fourth liquid water-rich by-products form part of the first liquid water-rich by-product, wherein at least part of the first water-rich by-product is recycled to step c).

12. The process according to claim 1, wherein the recycled liquid water-rich by-product contains at least 1 wt % oxygenates, based on the total recycled liquid water-rich by-product.

13. The process according to claim 1, further comprising, between steps (e) and (g) the step of
(f) separating sulphur compounds, carbon dioxide and other possible impurities from the shifted gas to obtain a purified synthesis gas.

14. The process according to claim 1, wherein only part of the synthesis gas is subjected to step (e) and wherein the remaining synthesis gas, which gas by-passes step (e), is combined with the shifted synthesis gas, optionally after performing a separate step (f) on both the shifted gas and the remaining gas, to obtain a combined synthesis gas effluent having a modified hydrogen to carbon monoxide molar ratio as feedstock for step (g).

15. The process according to claim 1, wherein step (h) comprises
h1) reacting part or all of the oxygenate effluent of step (g) and an olefinic co-feed, in the presence of an oxygenate conversion catalyst comprising at least 50 wt %, based on total molecular sieve in the oxygenate conversion catalyst, of a molecular sieve having one-dimensional 10-membered ring channels, to prepare an olefinic reaction effluent a second liquid water-rich by-product, wherein the olefinic co-feed comprises less than 10 wt % of C5+ hydrocarbon species;
h2) fractionating the olefinic reaction effluent to obtain at least a light olefinic fraction comprising ethylene, a heavier olefinic fraction comprising C4 olefins and less than 10 wt % of C5+ hydrocarbon species, and the second liquid water-rich by-product;
h3) recycling at least part of the heavier olefinic fraction obtained in step h2) as recycle stream to step h1), to form at least part of the olefinic co-feed; and
h4) withdrawing at least part of the light olefinic fraction as olefin-containing product.

16. A process to prepare a gasoline product from a solid carbonaceous feedstock by performing the steps of
(a) feeding an oxygen comprising gas and the carbonaceous feedstock to a burner firing into a reactor vessel, which burner is positioned horizontal,
(b) performing a partial oxidation of the carbonaceous feedstock in said burner to obtain a stream of hot synthesis gas and a liquid slag,
(c) cooling the hot synthesis gas by direct contacting with a liquid water-containing cooling medium,
(e) performing a water shift reaction on at least part of the synthesis gas, to obtain a synthesis gas effluent,
(g) performing an oxygenate synthesis using the synthesis gas effluent of step (e), to obtain a methanol and/or dimethylether containing oxygenate effluent and a first liquid water-rich by-product, and
(h) converting the oxygenate effluent to a gasoline product and a second liquid water-rich by-product,
wherein at least part of the first and/or second liquid water-rich by-product is used in step c), forming at least part of the liquid water-containing cooling medium.
